(19) United States
(12) Patent Application Publication
(10) Pub. No.: US 2026/0058000 A1
(43) Pub. Date: Feb. 26, 2026

(54) SYSTEM AND METHOD FOR ANONYMIZING IMAGES FOR USE IN OBSERVATION IN A HEALTHCARE SETTING

(71) Applicant: AvaSure, LLC, Belmont, MI (US)

(72) Inventors: Sit Soe, Battle Creek, MI (US); James Zampa, Evanston, IL (US); Dan William Michaels, Belmont, MI (US); Alejandro Alemany, Jackson, WY (US); Tristan Rogers, Grand Rapids, MI (US); Dimitri Angelov, Durham, NC (US); Adam Jamison Whitmore, Caledonia, MI (US); Brian Meinke, Rockford, MI (US); Walter Martin Eadelman, Belmont, MI (US)

(73) Assignee: AvaSure, LLC, Belmont, MI (US)

(21) Appl. No.: 19/169,640

(22) Filed: Apr. 3, 2025

Related U.S. Application Data
(60) Provisional application No. 63/686,490, filed on Aug. 23, 2024.

(51) Int. Cl. G16H 30/20; G06F 21/62; G06T 5/50; G06V 10/25; G16H 30/40

(52) U.S. Cl. CPC G16H 30/20; G06F 21/6245; G06T 5/50; G06V 10/25; G16H 30/40

(57) ABSTRACT

A method for anonymizing images for use in training learning models used in healthcare observation, wherein the images can be used as input data for the learning models, includes steps of collecting a real-time image from an image capturing device, analyzing the real-time image to identify specific regions of the real-time image that contain identifying information, determining physical characteristics of the specific regions, anonymizing the specific regions to obfuscate the identifying information to define an anonymized savable image that includes anonymized replicas of the physical characteristics of the specific regions, and storing the anonymized savable image within a database.

### CROSS-REFERENCE TO RELATED APPLICATION

This application claims priority to and the benefit under 35 U.S.C. § 119(e) of U.S. Provisional Patent Application No. 63/686,490, filed on Aug. 23, 2024, entitled SYSTEM AND METHOD FOR ANONYMIZING REAL-TIME AUDIOVISUAL STREAMS FOR HEALTHCARE, the entire disclosure of which is hereby incorporated herein by reference.

### FIELD OF THE DISCLOSURE

The present invention generally relates to observation and monitoring systems within a healthcare setting, and more specifically, a system and method for anonymizing data including images, video, and audio related to a healthcare setting. The anonymized data can be used for conducting healthcare observation and for creating a library of anonymized data that can be used for feeding into learning models for both analysis, training, and refining. The anonymized video can also be used for training and education purposes within a healthcare setting.

### BACKGROUND OF THE DISCLOSURE

Within many hospital and healthcare settings, video observation is used to monitor the position and status of a patient with respect to a healthcare space. This is frequently used in conjunction with an on-site or off-site observer, typically a human, that monitors a particular image along with a number of other images. Because of certain legal requirements and laws, storage of these videos must be done with the utmost care to prevent the unauthorized storage and/or release of patient information.

### SUMMARY OF THE DISCLOSURE

According to one aspect of the present disclosure, a method for anonymizing images for use in training learning models used in healthcare observation, wherein the images can be used as input data for the learning models, includes steps of collecting a real-time image from an image capturing device, analyzing the real-time image to identify specific regions of the real-time image that contain identifying information, determining physical characteristics of the specific regions, anonymizing the specific regions to obfuscate the identifying information to define an anonymized savable image that includes anonymized replicas of the physical characteristics of the specific regions, and storing the anonymized savable image within a database.

According to another aspect of the disclosure, a method for anonymizing images for use in healthcare observation, training of learning models, and for use for feeding as an input to learning models includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, storing the savable image within an anonymized database, categorizing the savable image within the anonymized database based on training categories, and inputting the savable image into machine learning models that correspond to the training categories, respectively, to evaluate subsequent video streams.

According to yet another aspect of the disclosure, a method for anonymizing images for use in training machine learning models for use in healthcare observation includes steps of collecting a real-time video stream from an image capturing device, selecting a representative image from the real-time video stream, analyzing the representative image to identify specific regions of the representative image that contain identifying information, removing data within the specific regions, placing replacement data into areas where the data was removed, thereby obfuscating the identifying information to define a savable image, wherein the replacement data is at least partially derived from image data that is located within the representative image and outside of the specific regions, storing the savable image within an anonymized database, categorizing the savable image within the anonymized database based on training categories, wherein the training categories include patient safety, staff safety, medical diagnosis, facility and staff operational efficiency, and false alarms, and training respective machine learning models related to the training categories using the savable image.

According to another aspect of the present disclosure, a method for anonymizing images for use in healthcare observation includes steps of collecting a real-time image from an image capturing device, analyzing the real-time image to identify specific regions of the real-time image that contain identifying information, determining characteristics of the specific regions, anonymizing the specific regions to obfuscate the identifying information to define a savable image, and storing the savable image within an anonymized image or video database or file storage library.

According to another aspect of the present disclosure, a method for anonymizing images for use in healthcare observation includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, applying the replacement images to the remaining images of the real-time video stream to define a savable video, and storing the savable video within an anonymized database.

According to yet another aspect of the present disclosure, a method for creating a database for use in training individuals for healthcare observation includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, applying the replacement images to the remaining images of the real-time video stream to define a savable video, storing the savable video within an anonymized database, and categorizing the anonymized database based on training categories.

According to yet another aspect of the present disclosure, a method for creating a database for use in training AI machine learning models for healthcare use cases related to patient safety, staff safety, medical diagnosis, and facility and staff operational efficiency includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, applying the replacement images to the remaining images of the real-time video stream to define a savable video, storing the savable video within an anonymized database, and categorizing the anonymized database based on training categories, then using these images or video to train all appropriate models while protecting the identity of people within the original captured images.

According to yet another aspect of the present disclosure, a method for creating a database for use in training AI machine learning models for healthcare use cases related to patient safety, staff safety, medical diagnosis, and facility and staff operational efficiency includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, applying the replacement images to the remaining images of the real-time video stream to define a savable video, and inputting the anonymized video directly to a single or multitude of AI machine learning models that will use the information to detect information appropriate to the purposes of the AI machine learning models.

According to yet another aspect of the present disclosure, a method for creating a database for use in training AI machine learning models for healthcare use cases related to patient safety, staff safety, medical diagnosis, and facility and staff operational efficiency includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, storing the savable images within an anonymized database, and categorizing the anonymized database based on training categories, then using these images to train all appropriate models while protecting the identity of people within the original captured images.

According to yet another aspect of the present disclosure, a method for creating a database for use in training AI machine learning models for healthcare use cases related to patient safety, staff safety, medical diagnosis, and facility and staff operational efficiency includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, and inputting the anonymized images directly to a single or multitude of AI machine learning models that will use the information to detect information appropriate to the purposes of the AI models.

According to yet another aspect of the present disclosure, a method for creating a database for use in training AI machine learning models for healthcare use cases related to patient safety, staff safety, medical diagnosis, and facility and staff operational efficiency includes steps of collecting a real-time audio stream from an audio capturing device, selecting an audio segment from the real-time audio stream, applying filters to the audio to anonymize the voice characteristics, detecting specific words from the audio stream that represent protected health information and replacing with appropriate but different words to obfuscate the identifying information to define a savable audio segment, and inputting the anonymized audio segment directly to a single or multitude of AI machine learning models that will use the information to detect information appropriate to the purposes of the AI machine learning models.

According to yet another aspect of the present disclosure, a method for creating a database for use in training AI machine learning models for healthcare use cases related to patient safety, staff safety, medical diagnosis, facility and staff operational efficiency includes steps of collecting a real-time audio stream from an audio capturing device, selecting an audio segment from the real-time audio stream, applying filters to the audio to anonymize the voice characteristics, detecting specific words from the audio stream that represent protected health information and replacing with appropriate but different words to obfuscate the identifying information to define a savable audio segment, and inputting the anonymized audio segment directly to a single or multitude of AI machine learning models that will use the information to detect information appropriate to the purposes of training the AI machine learning models.

These and other features, advantages, and objects of the present disclosure will be further understood and appreciated by those skilled in the art by reference to the following specification, claims, and appended drawings.

### BRIEF DESCRIPTION OF THE DRAWINGS

In the drawings:

FIG. 1 is a schematic perspective view of a healthcare setting showing placement of a patient monitoring device within the healthcare space;

FIG. 2 is a schematic perspective view of a healthcare setting showing a patient monitoring device used in conjunction with the patient and other occupants within the healthcare setting;

FIG. 3 is a schematic diagram illustrating an observer monitoring a number of real-time images;

FIG. 4 is a schematic diagram illustrating an observer utilizing a training and education module that includes at least one anonymized image created by an aspect of the anonymizing system described herein;

FIG. 5 is a schematic diagram showing an aspect of the anonymizing system that converts a real-time image into a savable image that has personal information obfuscated from view;

FIG. 6 illustrates a real-time image (a) and an anonymized savable image (b) that can be used for education and training purposes;

FIG. 7 illustrates a real-time image (a) and an anonymized savable image (b) that can be used for education and training purposes;

FIG. 8 illustrates a real-time image (a) and an anonymized savable image (b) that can be used for education and training purposes;

FIG. 9 illustrates a real-time image (a) and an anonymized savable image (b) that can be used for education and training purposes;

FIG. 10 is a linear flow diagram illustrating a method for anonymizing images for use in healthcare observation;

FIG. 11 is a linear flow diagram illustrating a method for anonymizing images for use in healthcare observation;

FIG. 12 is a linear flow diagram illustrating a method for creating an anonymized database of anonymized videos for use in training and education-related healthcare observation;

FIG. 13 is a flow diagram illustrating an aspect of the device that utilizes anonymized video and anonymized audio for creating and using a machine learning model that assists in patient observation in a healthcare setting;

FIG. 14 is a flow diagram illustrating an aspect of the device that utilizes anonymized video and anonymized audio for creating and using a machine learning model that assists in patient observation in a healthcare setting;

FIG. 15 is a flow diagram illustrating an aspect of the device for anonymizing images for use in training machine learning models for use in healthcare observation;

FIG. 16 is a flow diagram illustrating an aspect of the device for anonymizing images for use in training machine learning models for use in healthcare observation; and

FIG. 17 is a flow diagram illustrating an aspect of the device for anonymizing images for use in training machine learning models for use in healthcare observation.

The components in the figures are not necessarily to scale, emphasis instead being placed upon illustrating the principles described herein.

### DETAILED DESCRIPTION

As required, detailed embodiments of the present disclosure are disclosed herein; however, it is to be understood that the disclosed embodiments are merely exemplary of the invention that may be embodied in various and alternative forms. The figures are not necessarily to a detailed design; some schematics may be exaggerated or minimized to show function overview. Therefore, specific structural and functional details disclosed herein are not to be interpreted as limiting, but merely as a representative basis for teaching one skilled in the art to variously employ the present invention.

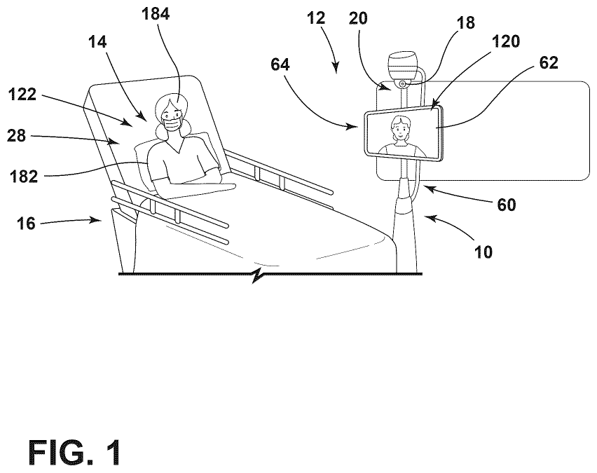

For purposes of description herein, the terms “upper,” “lower,” “right,” “left,” “rear,” “front,” “vertical,” “horizontal,” and derivatives thereof shall relate to the concepts as oriented in FIG. 1. However, it is to be understood that the concepts may assume various alternative orientations, except where expressly specified to the contrary. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

The present illustrated embodiments reside primarily in combinations of method steps and apparatus components related to a system and method for anonymizing real-time images, video streams and audio for use in observation and training in healthcare settings and for developing and building a database of anonymized data that can be used by an AI machine learning model for ambient monitoring used to assess and predict the status of a healthcare space and the patients and occupants therein. Accordingly, the apparatus components and method steps have been represented, where appropriate, by conventional symbols in the drawings, showing only those specific details that are pertinent to understanding the embodiments of the present disclosure so as not to obscure the disclosure with details that will be readily apparent to those of ordinary skill in the art having the benefit of the description herein. Further, like numerals in the description and drawings represent like elements.

As used herein, the term “and/or,” when used in a list of two or more items, means that any one of the listed items can be employed by itself, or any combination of two or more of the listed items, can be employed. For example, if a composition is described as containing components A, B, and/or C, the composition can contain A alone; B alone; C alone; A and B in combination; A and C in combination; B and C in combination; or A, B, and C in combination.

In this document, relational terms, such as “first” and “second,” “top” and “bottom,” and the like, are used solely to distinguish one entity or action from another entity or action, without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

As used herein, the term “about” means that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art. When the term “about” is used in describing a value or an end-point of a range, the disclosure should be understood to include the specific value or end-point referred to. Whether or not a numerical value or end-point of a range in the specification recites “about,” the numerical value or end-point of a range is intended to include two embodiments: one modified by “about,” and one not modified by “about.” It will be further understood that the end-points of each of the ranges are significant both in relation to the other end-point, and independently of the other end-point.

The terms “substantial,” “substantially,” and variations thereof as used herein are intended to note that a described feature is equal or approximately equal to a value or description. For example, a “substantially planar” surface is intended to denote a surface that is planar or approximately planar. Moreover, “substantially” is intended to denote that two values are equal or approximately equal. In some embodiments, “substantially” may denote values within about 10% of each other, such as within about 5% of each other, or within about 2% of each other.

As used herein the terms “the,” “a,” or “an,” mean “at least one,” and should not be limited to “only one” unless explicitly indicated to the contrary. Thus, for example, reference to “a component” includes embodiments having two or more such components unless the context clearly indicates otherwise.

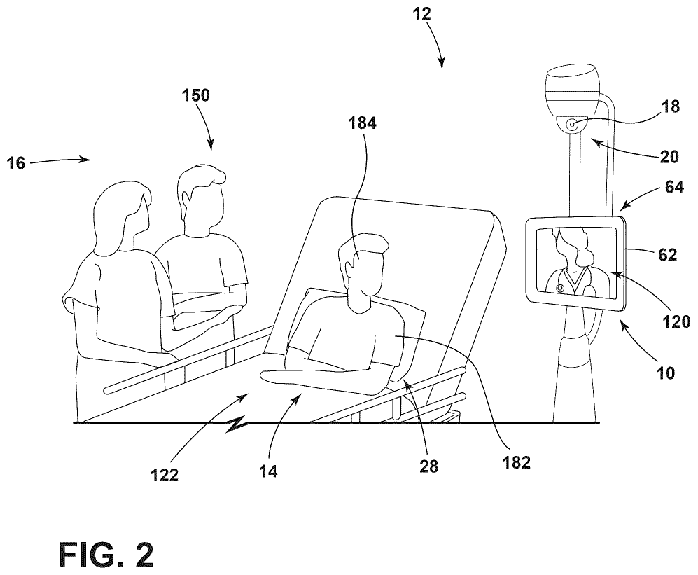

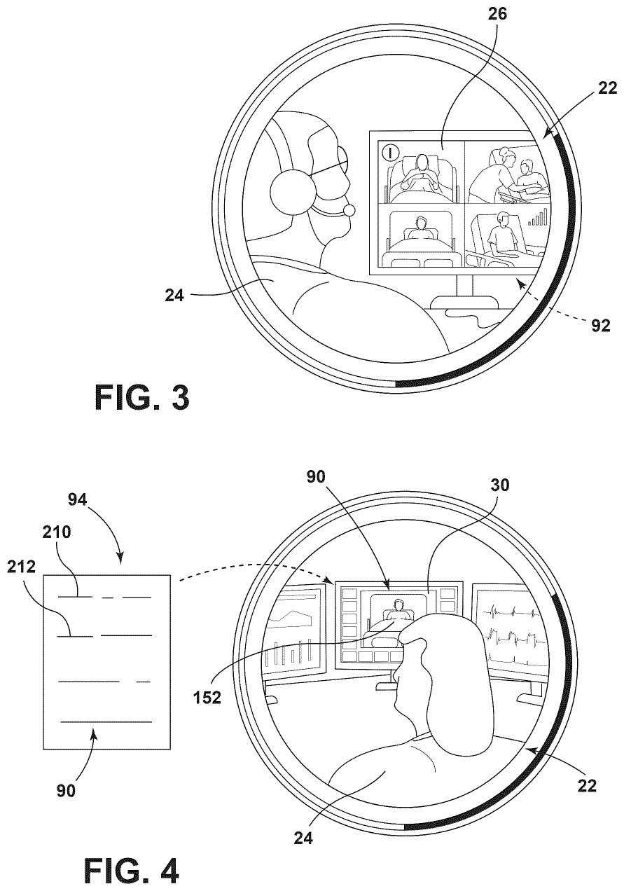

Referring to FIGS. 1-9, reference numeral 10 generally refers to a monitoring device that can be used within a healthcare setting 12 for monitoring the movements and general status of a patient 14 staying within a particular healthcare space 16. The monitoring device 10 includes at least one image capturing device 18, such as a camera, LIDAR, radar, infrared camera, and other similar image capturing devices. The monitoring device 10 can also include one or more microphones 20 for capturing audio within the healthcare space 16. The monitoring device 10 captures real-time audio and real-time images 26 within the healthcare space 16 for use in observing the motions and status of the patient 14, or used for automated ambient monitoring by a learning model 320, such as an artificial intelligence (AI) learning model or machine learning model. This information is transmitted to an observing device 22 that can be located on site or at an off-site location. An observer 24 monitors a real-time image 26, such as a video feed, from the monitoring device 10 to ensure that the patient 14 is in a stable condition 28. Additionally, the learning model 320 can provide automated alerts when a particular healthcare-related event, grouping of events, item, or grouping of items is detected. The observer 24 can be provided with certain criteria for observing the patient 14. Such criteria can include fall risk, consistent patient movement, auditory cues, elopement, equipment monitoring, and other similar criteria, as described herein. Based on this criteria, the observer 24 monitors the patient 14 to determine whether the patient 14 is in a stable condition 28, or whether some form of intervention is needed. Where an intervention is needed, the observer 24 can initiate any one of various interventions. These interventions can include, but are not limited to, auditory interactions by the observer 24, sending an alert to a nurse device and/or a nurse station, sounding an auditory alarm, and other similar interventions.

Referring again to FIGS. 1-9, as described herein, the real-time images 26 can be used as training images 30 for training learning models 320 regarding the detections of healthcare-related events using the criteria, so long as these real-time images 26 are modified to remove information that could be used to identify the patient 14 and other similar sensitive or personal information. As described herein, the real-time images 26 can be used as training images 30 for training of learning models 320. In certain aspects of the device, it is contemplated that the real-time images 26 can be used as an education tool for training observers 24. The use of the real-time images 26 is possible, so long as these real-time images 26 are modified to remove information that could be used to identify the patient 14 and other similar sensitive or personal information contained or derivable from the real-time images 26.

Referring again to FIGS. 1-4, the monitoring device 10 can include various user interface features 60. Such user interface features 60 can include a monitor 62, teleconference equipment 64, input devices, speakers, and other similar components. The monitoring device 10 can be a mobile device that can be moved from one healthcare space 16 to another based upon the characteristics of the particular patient 14. It is also contemplated that the monitoring device 10 can be a built-in unit installed within the healthcare space 16, such as on the ceiling, on a wall, or other similar area of the healthcare space 16.

According to various aspects of the device, use of the real-time images 26 that are taken by the monitoring device 10 and delivered to the observing device 22 must be utilized with care. Certain laws and legal requirements, such as HIPAA (Health Insurance Portability and Accountability Act) in the United States and similar laws of other countries and jurisdictions, prevent storage of these real-time images 26 unless proper consent is obtained in advance. These laws are used to protect the confidentiality of patients and their families so that this information cannot be improperly used. Because of these requirements, training for machine learning models 320 and for observers 24 presents a challenge. In certain conditions, actors, animations, or other prepared videos can be utilized to recreate certain monitoring conditions with which an observer 24 may be faced. These recreations can be unrealistic and may not necessarily present real-world conditions that the observer 24 will be facing when observing one or more real-time images 26. Because of the potentially unrealistic nature of these recreations, training of learning models 320 can be limited.

Referring again to FIGS. 1-9, for training a learning model 320 using more accurate representations of observable conditions, an anonymizing system 92 and method for creating a library or other database 94 of anonymized savable images 90 is disclosed herein. The anonymizing system 92 described herein allows for the creation of a library of anonymized savable images 90 that removes all Patient Health Information (PHI) 120 and Patient Identifying Information (PII) 122 from being saved and/or disclosed. To accomplish the creation of savable images 90, the real-time images 26 are anonymized using the anonymizing system 92 to obfuscate this sensitive information so that no PHI 120 or PII 122 is being stored or disclosed. Once the real-time images 26 are turned into anonymized savable images 90, the savable images 90 can be stored within the database 94 for later use in training, education, or for use with learning models 320 and for training these learning models 320. Within this disclosure, the term “obfuscate”, or “obfuscation” includes actions, methods and operations that render certain data, such as PHI 120 and PII 122, irretrievable. This obfuscation may include removing, distorting, supplanting, overlaying, or otherwise rendering not visible, PHI 120 and PII 122.

As exemplified in FIGS. 1-9, with respect to the patient 14 and other occupants 150 of the healthcare space 16, certain aspects of the anonymizing system 92 can use a “deepfake” technology for replacing physical characteristics 350 of the patient 14 and other occupants 150 included within the real-time image 26 with replacement images 152 that contain no PHI 120 or PII 122. With respect to these portions of the real-time image 26, this sensitive information can be obfuscated or removed by the anonymizing system 92 to blur, black out, redact, or otherwise render irretrievable other sensitive information within the real-time image 26 of the healthcare space 16.

Referring again to FIGS. 1-9, according to the various aspects of the device, the anonymizing system 92 captures a real-time image 26 that is delivered to an observer 24 in real time or near real time. Typically, when an observer 24 observes some event that may warrant intervention, the observer 24 or other user can designate the real-time image 26 for further analysis to run the anonymizing system 92. The selected real-time image 26 will typically include some event, occurrence, or situation that may be useful in training one or more machine learning models 320. Using the anonymizing system 92, this real-time image 26 is modified to remove the PHI 120 and PII 122 from the video, or other real-time image 26, and replace this information with replacement images 152 that render irretrievable the PHI 120 and PII 122 so that none of this sensitive information is visible or obtainable. The analysis of the real-time image 26 and anonymization of the real-time image 26 takes place before the real-time image 26 is stored so that no PHI 120 or PII 122 is stored during the process of anonymizing a particular real-time image 26. Only after the real-time image 26 has been properly anonymized into a savable image 90 is the savable image 90 stored within the database 94 for later use in training and education of observers 24.

It is contemplated that, during analysis to remove the PHI 120 and PII 122, the real-time image 26 can be stored for a short period of time within a volatile memory, data buffering system, or other memory that is frequently overwritten with new data, to conduct the analysis of the real-time image 26. Only after the PHI 120 and the PII 122 have been removed is the real-time image saved in a long-term storage, as described herein.

In certain aspects of the device, the analysis and anonymization of a particular real-time image 26 and/or captured audio can occur in real time such that the savable image 90 and/or audio segment is created contemporaneously with the image capturing device 18 and microphone 20 collecting the real-time image 26 and real-time audio. The analysis and anonymization of the real-time video can also be conducted by the anonymizing system 92 shortly after the real-time image 26 is taken. In such an aspect of the device, the real-time image 26 is not stored but is set aside within a volatile memory, such as a Random Access Memory (RAM) or buffering system, so that the analysis and anonymization can take place. Where the volatile memory is used, it is typical that the volatile memory is frequently overwritten or deleted such that no permanent storage of data can take place. Only after the PHI 120 and the PII 122 is rendered irretrievable and the savable image 90 is created, as described herein, is the saved image stored within a non-volatile memory, such as a cloud-based storge or a Read Only Memory (REM), for permanent or semi-permanent storage. It is also contemplated that the analysis and anonymization of a particular image or video can occur shortly after the real-time image 26 is created. In such an aspect of the device, the real-time image 26 may be buffered into a packet of data for delivery to a separate location. During this process of gathering and transporting video, the analysis and anonymization of the video can take place so that the real-time image 26 is not stored in a non-volatile memory.

Referring again to FIGS. 1-9, the PHI 120 and PII 122 that is being anonymized within the real-time images 26 can include various portions of a particular real-time image 26. These specific regions 180 can include the entire body 182 and facial features 184 of the patient 14, the body 182 and facial features 184 of occupants 150 within the healthcare space 16, and text 186 within the healthcare space 16. Text 186 can include information such as room number, medical notations, names, dates, and other similar identifying information or healthcare information. PHI 120 can include medications being prescribed, names of nurses and attending physicians, visible diagnoses or treatment regimens, and other similar healthcare information. Additionally, reflections of the PHI 120 and PII 122 on reflective surfaces may also be visible and in need of obfuscation.

As a particular real-time image 26 is being analyzed for anonymization, the analysis includes a review of the entire real-time image 26 to determine specific regions 180 that may contain PHI 120 and PII 122 that is to be obfuscated. As described herein, the image is not stored within the database 94 until all of the PHI 120 and PII 122 has been properly obfuscated. Once the specific regions 180 have been identified, the process of anonymizing the specific regions 180 can take place.

Referring again to FIGS. 1-9, the process of obfuscating a real-time image 26 can be accomplished through various masks 250, or filters, that evaluate the pixels 252 and data of the real-time image 26 in order to ascertain which portions of the real-time image 26 should be obfuscated. These masks 250 can include a series of filters that progressively finetune the obfuscation process in order to select those portions of the real-time image 26 that contain PHI 120 or PII 122, or both. By way of example, and not limitation, a first filter, or mask, segments the pixels 252 of the real-time image 26 to determine which pixels 252 correspond to a person 260, such as a patient 14 or an occupant 150, and which pixels 252 correspond to areas and portions that are not a person. This initial mask can utilize various mechanisms for evaluating the pixels 252 of the real-time image 26. In one exemplary instance, shapes that correspond to orthogonal corners 254 or flat surfaces 256 may typically correspond to furniture, structural surfaces, and other fixtures 258 within the real-time image 26. Those silhouettes 282, boundaries, and other profiles that include more irregular shapes, or shapes corresponding to features of the human body 182, can be selected for further analysis as being indicative of a person 260. Again, information relating to a person 260 in the real-time image 26 is likely to require some level of obfuscation.

Referring again to FIGS. 1-9, a subsequent mask 250 can be applied to the filtered pixels 252 that correspond to a person 260 from the real-time image 26. In this subsequent mask 250, the refined data can be evaluated to determine which pixels 252 should be obfuscated because they contain at least one of PHI 120 and PII 122. This can be accomplished through a machine learning model 320 that is able to evaluate the pixels 252 and other data of the real-time image 26 that is attained through use of the initial mask 250 to determine which portions of the pixels 252 should be obfuscated. Using multiple masks 250, the pixels 252 can be segmented into pixels 252 that do not require obfuscation, and pixels 252 that require additional analysis. Each mask 250 that is used can refine this process to arrive at those pixels 252 that correspond to PHI 120 and PII 122. As described herein, the data to be obfuscated typically includes facial features 184, exposed portions of the human body 182, clothing, and other portions of the pixels 252 that are indicative of the human body 182. This process of refining the data through the use of one or more masks 250 can be referred to as segmentation. In this process of segmentation, the real-time image 26 is segmented into areas to be obfuscated and areas that do not need to be obfuscated. The segmentation includes separating the real-time image 26 into pixels 252 and other data that corresponds to PHI 120 or PII 122, and data that corresponds to things other than PHI 120 or PII 122. Based upon the outcome of this segmentation process, the specific regions 180 can be obfuscated and the remaining portions of the real time image 26 can remain unchanged.

It is contemplated that additional masks 250 can be applied to the real-time image 26 to ascertain which portions outside of a person may also need to be obfuscated. As described herein, this information can include text, screens or monitors, documents, and other portions of the real-time image 26 that may need to be obfuscated to prevent the saving of PHI 120 or PII 122, as well as information that may lead to the recovery of these types of information.

Once those portions of the real-time image 26 have been segmented and the specific regions 180 are identified, the real-time image 26 can undergo the obfuscation process. In certain aspects of the device, the obfuscation can include extracting data from the specific regions 180 of the real-time image 26 that include the PHI 120 and the PII 122. During this process of extraction, corresponding replacement regions 280 are generated for filling in the sections of data left by the removal of the specific regions 180 of the real-time image 26. Accordingly, as the specific regions 180 are identified, replacement regions 280 can be generated for adding to the real-time image 26. The specific regions 180 include silhouettes 282 and internal boundaries 284 that correspond to profiles of portions of a human body 182. The silhouette 282 can correspond to the outline of the body 182 and the outline of certain portions of the body 182. The internal boundaries 284 can include finer details, such as outlines of facial features 184. The replacement images 152 can include anonymized replicas 286 of the specific regions 180. These anonymized replicas 286 can include similar silhouettes 288 and similar boundaries 290 that are used to replace the specific regions 180 that have been removed from the real-time images 26.

The replacement regions 280 having the similar silhouette 288 and similar boundaries 290 show the positioning of features of the human body 182, without providing identifying information relating to a particular individual within the real-time image 26. These replacement regions 280 are then inserted into the sections of data left by the removal of the specific regions 180 of the real-time image 26. By removing the specific regions 180 of the real-time image 26, the resulting savable image 90 can be modified to redact or delete information corresponding to PHI 120 or PII 122. Accordingly, the resulting savable image 90 does not include these types of information.

According to various aspects of the device, the specific regions 180 of the real-time image 26 can also be overlaid or overwritten by the replacement regions 280 described herein. By overwriting the specific regions 180 with the replacement regions 280, PHI 120 and PII 122 are removed from the real-time image 26 to derive the savable image 90. In each of these processes of anonymization and obfuscation, the goal is to produce a savable image 90 that does not include PHI 120 and/or PII 122. Additionally, the savable image 90 is configured such that any PHI 120 and PII 122 from the original real-time image 26 is irretrievable and otherwise unattainable through use of the savable image 90.

According to the various aspects of the device, the real-time images 26 that are used for creating the savable image 90 can be selected from a real-time video stream. The real-time images 26 can be randomly selected representative images 310 within a portion of the real-time video stream. These randomly selected representative images 310 are selected with at least a patient 14 included within the real-time video stream. Additional occupants 150, such as healthcare staff and visitors, can be present within the selected real-time image 26 to define the representative image 310. It is also contemplated that the real-time image 26 can be selected from a video feed that includes an identified event. Such an identified event can be a confirmed healthcare issue, such as a patient fall, an attempted elopement, interfering with hospital equipment, and other similar events. The identified event can also be a confirmed false alarm. A confirmed false alarm may be an event where an alarm was sounded, but no event resulted after the activation of the alarm. Once the real-time image 26 is selected, the process of anonymization and obfuscation of the real-time image 26 can be performed to arrive at the savable image 90.

According to the various aspects of the device, the identified event can be a healthcare issue or a potential healthcare issue. These events can include a patient getting up from a bed, a patient reaching for an object, a patient walking towards the door, and other similar movements and occurrences within the video feed from which the real-time images 26 are selected. These events typically relate to healthcare training categories 210 that can include, but are not limited to, patient events, staff events, medical diagnoses, facility and staff operational efficiency, false alarms, combinations thereof, and other similar categories that can be used for evaluating the savable image 90.

According to the various aspects of the device, as exemplified in FIGS. 1-9, the process of obfuscation, as described herein, is operated through use of one or more machine learning models 320 that can utilize artificial intelligence for evaluating the pixels 252 and other data of the real-time image 26 for determining the silhouette 282 and internal boundaries 284 of the specific regions 180 of the real-time image 26 that include PHI 120 or PII 122, or both.

In certain instances, after the machine learning models 320 have performed the obfuscation procedures, it is contemplated that a human can be utilized as a final check of the obfuscated image to determine whether all PHI 120 and/or PII 122 have been obfuscated from the real-time image 26. This human reviewer can then provide approval that the image is confirmed as a savable image 90 that can be stored within a database 94 for later use, as described herein.

Once the savable image 90 is generated, the savable image 90 can be labeled to include tags 330 that highlight certain features within the savable image 90. These features can include, but are not limited to, the identity of certain portions of the human body 182, certain movements of a person within the savable image 90 that are indicative of a particular identified event, objects within the savable image 90, and other similar tags 330. These tags 330 can be identifiers that can be used for training the machine learning model 320 for identifying indicators within a subsequent video stream related to a particular healthcare issue or information indicative of a false alarm.

According to the various aspects of the device, the replacement regions 280 that are placed within the real-time image 26 for generating the savable image 90 can be obtained through various sources. In an exemplary aspect of the device, the replacement regions 280 can be gathered from information contained within the real-time image 26. In this manner, colors within pixels 252 surrounding the specific region 180 of the real-time image 26 can be utilized for determining the color palette of the replacement regions 280 that are positioned within, or over, the specific regions 180 of the real-time image 26. In utilizing colors from within the real-time image 26, it is possible to generate replacement regions 280 having a boundary that may have a low level of contrast or a subtle contrast, referred to herein as a low contrast boundary 340, in relation to the area surrounding the replacement region 280 of the savable image 90.

Within a video feed, the area surrounding a patient 14 may have a low contrast boundary 340, such as the boundary between a hospital gown of the patient 14 and blankets and sheets on the patient's bed. Also, boundaries between individuals within the room may define a low contrast boundary 340, where individuals may be wearing similar colors of clothing. In events such as these, the boundary between individuals within the real-time image 26 may be a low contrast boundary 340.

By using information within the real-time image 26 to generate the replacement regions 280, the low contrast boundary 340 can be repeated and implemented within and around the replacement regions 280 that form a portion of the savable image 90. This low contrast boundary 340 can also be labeled with a tag 330 for use within a machine learning model 320 that utilizes the savable image 90 for evaluating a subsequent video stream. Using the savable image 90 having low contrast boundaries 340, the machine learning model 320 evaluating a subsequent video feed is better equipped to evaluate the boundaries between objects and individuals within the real-time image 26. In this manner, the machine learning model 320 is able to assess events and movement with respect to low contrast boundaries 340. Accordingly, the machine learning model 320 is better equipped to provide advance information within the machine learning model 320, and ultimately to an observer 24, for assessing events within the video feed and providing certain predictions. These predictors can be evaluated before a high contrast boundary around a patient 14 appears within the real time image 26, such as when a patient 14 leaves a hospital bed.

According to the various aspects of the device, the generated images that create the replacement regions 280 of the savable image 90 can also be pulled from public domain images, can be entirely generated using data within the real-time image 26, or combinations thereof. As described herein, these replacement images 152 are typically generated, at least in part, through information derived and obtained from the real-time image 26 itself to generate the low contrast boundaries 340 that may be present within the real-time image 26. As described herein, re-creating these low contrast boundaries 340 within the replacement regions 280 assists in training the machine learning model 320 to identify and evaluate these low contrast images within a subsequent video feed.

According to the various aspects of the device, information that can be extracted, or otherwise obfuscated from the real-time image 26 can include pixels 252 that are visible within the real-time image 26. Metadata can also be removed from the image. It is contemplated that certain information may be kept within the savable image 90. Where retained, such information can include, but is not limited to, the name of the healthcare facility, a particular location, a date, and other similar information. This information that may be saved in combination within the savable image 90 can be stored in a way that does not include PHI 120, PII 122, or information that can lead to the retrieval of PHI 120 or PII 122.

According to the various aspects of the device, where tags 330 are included within the savable image 90, the tags 330 can identify landmarks within, or training categories 210 of information within, the savable image 90. These training categories 210 can include, but are not limited to, patient events, staff events, medical diagnoses, facility and staff operational efficiency, false alarms, combinations thereof, and other similar categories that can be used for evaluating the savable image 90. These training categories 210 can also include types of motion within the real-time image 26. Because the real-time image 26 is a single frame, frames of a video feed before and after the real-time image 26 can also be evaluated for determining directions of movement and classifications of motion within the real-time image 26. These movements can be identified with a tag 330. These tags 330 can also be used to identify training categories 210 of items and surfaces within the real-time image 26 that relate to the patient 14 and/or a particular healthcare issue. These items can include a wall, a floor, furniture, equipment, and other similar objects and fixtures 258 within the real-time image 26.

As described herein, the savable images 90 can be saved according to corresponding training categories 210 within a database 94. These savable images 90 are categorized for use within one or more machine learning models 320. These machine learning models 320 can be applied for evaluating a subsequent video feed for assessing information within the subsequent video feed. The machine learning model 320 can compare the savable image 90, and the tags 330 contained therein, with information within the subsequent video feed. The machine learning model 320 can evaluate this information for determining matches that may be indicative of certain healthcare events that can be communicated to an off-site observer, a nurses' station, a healthcare facility, or other healthcare location.

In certain aspects of the device, the machine learning model 320 can be utilized for detecting occupants 150 within a particular room. Other machine learning models 320 can be utilized for detecting motion of a patient 14 within certain areas of a healthcare setting 12. Machine learning models 320 can also be utilized for evaluating the subsequent video feed for determining the occurrence of certain actions or movements by a patient 14 within a healthcare setting 12. Again, the machine learning model 320 compares the savable images 90 with the individual frames of the subsequent video feed for determining whether a healthcare event has occurred, or is about to occur, as well as whether the frames are indicative of a healthcare event not happening, such that activating an alarm would be a false alarm of these types of events.

The database 94 that stores the savable images 90 can be divided into training categories 210 that correspond to the various machine learning models 320 that are used to evaluate subsequent video feeds. Again, these machine learning models 320 can be dedicated to evaluate the subsequent video feed for assessing certain types of information and data within the subsequent video feed. These machine learning models 320 can also be configured to operate in combination. In this manner, the various machine learning models 320 can evaluate overlapping indicators to provide more robust and reliable information regarding certain events.

By way of example, and not limitation, one machine learning model 320 that is devoted to assessing patient movements can operate in combination with a machine learning model 320 that is meant to evaluate the motion of bedding and fabric on a patient's bed. In this exemplary aspect, these two machine learning models 320 can cooperate to determine that a patient 14 is moving toward the edge of the bed or about to get up from the bed.

The anonymization of the specific regions 180 can include blurring certain portions of the real-time image 26 that may contain text 186 or other information. Other specific regions 180 of the real-time image 26, such as the patient 14 and other occupants 150 within the room, can be replaced through deep fake images, animations, or other similar replacement images 152 that at least partially, and typically fully, replace the entire body 182 of the patient 14 or other occupant 150 in the healthcare space 16 with randomly selected replacement images 152, as described herein.

By using the replacement images 152, the motions and actions of the patient 14 and other occupants 150 within the healthcare space 16 can be documented and used for training machine learning models 320, and, in certain aspects of the device, for education purposes. Typically, these actions, motions, and other movements of the patient 14 and the occupants 150 serve as the basis for the training machine learning models 320 and to be included within the database 94. By replacing the images of the patient 14 from the real-time image 26 with replacement images 152, these movements of the patient 14 and occupants 150 can be stored for review by the machine learning model 320, or by an observer 24, without storing or disclosing sensitive information such as PII 122 and PHI 120.

According to the various aspects of the device, the replacement images 152 used to replace the body 182 of the patient 14 can include a single predefined body, such as that of an actor, animation, avatar, or other similar replacement body. It is also contemplated that the replacement images 152 can be pieced together to mimic an outline of the patient 14 so that the entire body 182 of the patient 14 can be obfuscated, without also eliminating other portions of the healthcare space 16 that may be relevant to the training of the machine learning model 320 or the education of observers 24. The replacement images 152 can be gathered from volunteers who authorized the use of their image and likeness, as well as other similar public domain images that can be utilized as replacement images 152. Components of these can be parsed and evaluated for matching profiles and used as the replacement images 152. Again, the replacement images 152 are used to obfuscate the identity of the patient 14 and the occupants 150 as well as information that can lead to the identity of the patient 14 and occupants 150, without obfuscating the movements of these individuals that may be useful in training and education of observers 24, as well as training a machine learning model 320 that can draw from anonymized data for assisting an observer 24 in monitoring a patient 14, or providing ambient observation of a patient 14.

According to various aspects of the device, the types of real-time images 26 that can be utilized in conjunction with the anonymizing system 92 and method can include still images, videos, three-dimensional images and videos, thermal images, and other similar images which may show varying degrees of PII 122 or PHI 120 within the image. It should be understood that the term “image” as used broadly within this disclosure is meant to not only include still images, but also include those types of images and videos described herein. The term “image” as used in this disclosure is also meant to include stand alone audio as well as audio that is captured along with stills and videos. Accordingly, the anonymization of audio is also contemplated where voices can be replaced, modulated, or otherwise obfuscated so that any PHI 120 or PII 122 is sufficiently obfuscated. When obfuscating audio files, care is also taken to obfuscate or eliminate any words or context that may include or indicate PHI 120 or PII 122 therein. In certain aspects of the device, the database 94 will store an anonymized transcript of the audio that can be used to train the AI machine learning models 320 for analyzing real-time images 26 for observation or for future anonymization of future real-time images 26.

A goal of the anonymizing system 92 described herein is to create anonymized savable images 90 and savable anonymized audio that can be used in building a database 94 for an AI machine learning model 320 to draw from to provide ambient monitoring of a healthcare space 16 to assess the status of a patient 14, occupants 150, and various fixtures 258 and equipment within the healthcare space 16. Another goal of the anonymizing system 92 described herein is to create training and education videos that can be utilized for training observers 24 and also providing continuing education to observers 24. In this manner, real-world situations that have actually occurred can be utilized in developing the database 94 for the machine learning model 320 to draw from as well as for training and educating observers 24. These real-world events can be used to make the machine learning model 320 and observers 24 more efficient at their task. Additionally, these real-world events provide a robust library of experience for common and unique events to the machine learning model 320 and the observers 24 so that fewer situations are encountered for which no training may have been provided to the machine learning model 320 or the observers 24.

According to various aspects of the device, as the anonymized savable images 90 are gathered, these anonymized savable images 90 are stored in the database 94. This database 94 can be divided into training categories 210 based on information that is contained within the savable image 90. By categorizing the savable images 90, dedicated machine learning models 320 can be utilized, where each machine learning model 320 using information related to a single category or a set of categories. In this manner, the categorized savable images 90 can be accessed by the AI machine learning model 320 for analyzing discrete portions of a subsequent real time image 26. The database 94 can be configured to store different training categories 210 of savable images 90, anonymized video, anonymized audio and other data that relate to different types of events which the AI machine learning model 320 and an observer 24 may encounter.

For human training and education, certain modules 212 can also include a randomized set of situations that the observer 24 can use as a training and education aid. Over time, additional anonymized savable images 90, audio and other data can be added to the database 94 to provide a robust library or catalog of anonymized savable images 90 that can be used by the AI machine learning model 320 or for training and education purposes.

According to the various aspects of the device, where a portion of a video feed is to be anonymized, such as a certain number of successive frames, identifying information within any specific regions 180 of a video stream can include static portions 230 and dynamic portions 232. These static portions 230 can include text 186 on a screen or document, and other portions of the image that may not move from one frame of the real time image 26 to the next. The dynamic portions 232 can include portions of the patient 14 and other occupants 150 of the healthcare space 16 that do move from one frame of the real time image 26 to the next. The system of anonymizing the static portions 230 and dynamic portions 232 of the real-time image 26 can be performed using the same methodology or different methodology. Typically, where a static portion 230 of the real-time image 26 is determined to contain PHI 120 or PII 122, those portions of each frame with the real-time image 26 will be anonymized in a similar fashion such that additional analysis may not be desired. For the dynamic portions 232 of the real-time image 26 that contain PHI 120 or PII 122, greater analysis may be required between individual frames for determining the scope and boundaries of the PHI 120 and PII 122 so that an appropriate replacement image 152 can be included for obfuscating the PHI 120 and PII 122 to create the savable image 90.

According to the various aspects of the device, once the anonymization is complete, the savable image 90 can be compressed such that the anonymized image becomes a final version that cannot be reverted back to its previous state, as constituted in the real-time image 26. Rather, once the obfuscation included within the anonymized image is complete, that information cannot be reversed such that the PHI 120 and PII 122 contained in the real-time image 26 becomes irretrievable.

According to various aspects of the device, in certain situations, it may be desirable to have a confirming step where a system, program, or individual reviews the anonymization of the savable image 90 to ensure that all PHI 120 and PII 122 has been properly and entirely obfuscated by the anonymizing system 92. This confirmation, as described herein, is conducted before the modified image is saved into the database 94. Where the anonymization is incomplete, the image can be rerun through the anonymizing system 92 or can be entirely discarded without being saved into a permanent memory or database 94. Only after confirmation of obfuscation of all PHI 120 and PII 122 within the real-time image 26 is completed is the video stored within the database 94 as a savable image 90 for later use in training and education.

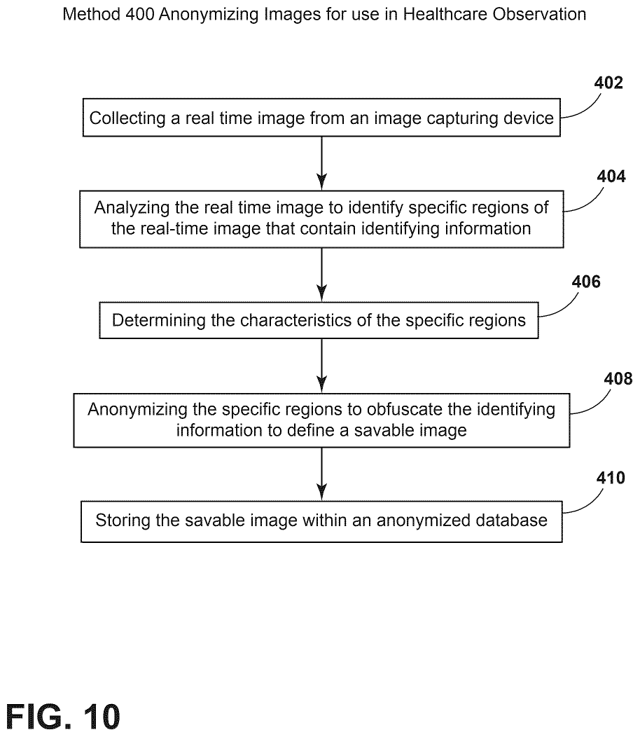

Referring now to FIGS. 1-10, having described various aspects of the anonymizing system 92, method 400 is disclosed for anonymizing images for use in healthcare observation. According to method 400, a real-time image 26 is collected from an image capturing device 18 (step 402). As described herein, collecting of the real-time image 26 occurs through the use of the monitoring device 10 that is placed within the healthcare space 16. The image can include graphic elements, auditory elements, and other layers of information that may be included within the real-time image 26. Once the real-time image 26 is collected, the real-time image 26 is analyzed to identify specific regions 180 of the real-time image 26 that contain identifying information (step 404). As part of this analyzing step, the characteristics of the specific regions 180 are determined (step 406). This determination can include what type of information is included within the specific region 180, such as dynamic portions 232 and static portions 230. Additionally, determining the characteristics can include assessing whether the information is related to the body 182 of a patient 14, some other occupant 150 within the room, text 186 within the real-time image 26, reflections off from reflective surfaces, audio information, and other similar characteristics. This analysis is typically performed by a machine learning model 320 that evaluates the real time image 26 to determine wherein PHI 120 and/or PII 122 may be present and in need of obfuscation. After these characteristics have been determined, method 400 includes a step 408 of anonymizing the specific regions 180 to obfuscate the identifying information to define a savable image 90. As described herein, this anonymizing step can include various levels of anonymizing, again, typically performed by a machine learning model 320. These levels can include an initial anonymization step, and subsequent confirmation steps of anonymizing the PII 122 and PHI 120. After the anonymizing step is complete, the savable images 90 are stored within an anonymized database 94 (step 410). As described herein, this anonymized database 94 is utilized for training machine learning models 320. It can also be used for education purposes with respect to observers 24, nurses, and other individuals that may be tasked with observing real-time images 26 of healthcare spaces 16 for determining the status of the patient 14 and other occupants 150 within a particular healthcare space 16.

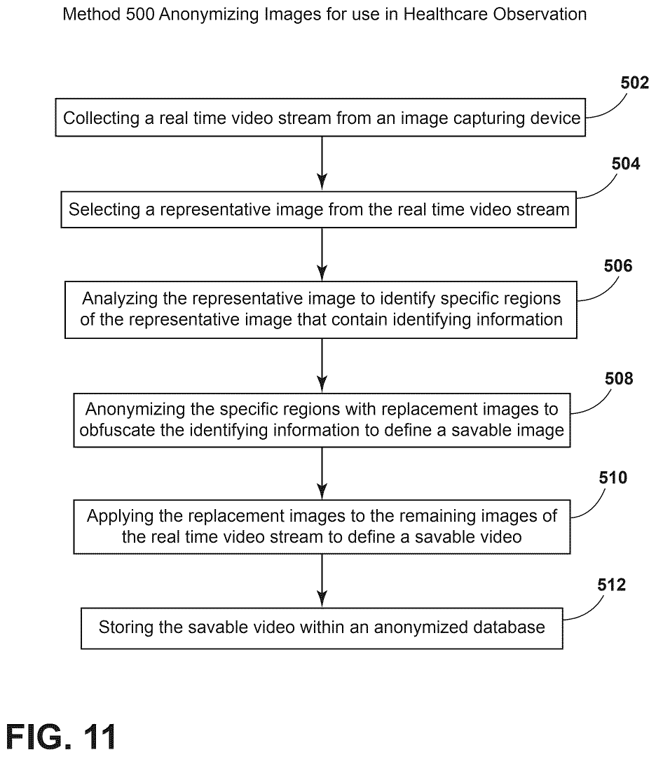

Referring now to FIGS. 1-9 and 11, method 500 is disclosed for anonymizing images for use in healthcare observation. According to method 500, step 502 includes collecting a real-time image 26, such as a video stream, from an image capturing device 18. Once collected, a representative image 310 from the real-time video stream is selected (step 504). This step 504 of selecting a representative image 310 can be used for providing a base image related to the specific regions 180 that may require obfuscating using the anonymizing system 92. Once the representative image 310 is selected, the representative image 310 is analyzed, typically using a machine learning model 320, to identify the specific regions 180 therein that may contain identifying information, such as PII 122 and PHI 120 (step 506). Once the analysis of the representative image 310 is complete and the specific images have been identified, the specific regions 180 are anonymized, again using a machine learning model 320, with replacement images 152 to obfuscate the identifying information and to define a savable image 90 (step 508). The replacement images 152 that were disposed within the representative image 310 are then applied to the remaining images of the real-time video stream to define a savable image 90 in the form of a savable video (step 510). As with other anonymizing steps described herein, it may be desirable to utilize one or more confirming steps to confirm that the PHI 120 and PII 122 within a particular video stream has been properly anonymized and obfuscated. Again, these confirming steps occur before the savable video is stored within the anonymized database 94 (step 512).

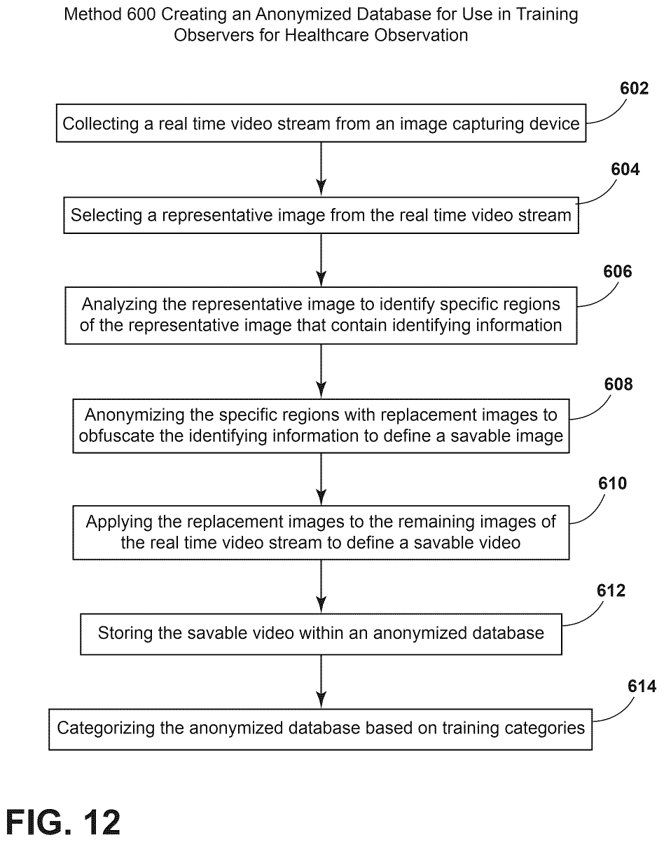

Referring now to FIGS. 1-9 and 12, method 600 is disclosed for creating an anonymized database 94 for use in training individuals for healthcare observation. According to method 600, a step 602 includes collecting a real-time image 26, such as a video stream, from an image capturing device 18. Once collected, a representative image 310 from the real-time video stream is selected (step 604). This selection can be at random, or can be a discriminate selection, as described herein. In certain aspects of the device, the representative image 310 may or may not be desired depending upon the efficiency and type of analysis that must be conducted with respect to the real-time video stream and the level of replacement images 152 that must be included within the real-time video. Once the representative image 310 has been selected, the representative image 310 is analyzed, using a machine learning model 320, to identify specific regions 180 of the representative image 310 that contain identifying information, such as PII 122 and PHI 120 (step 606). The specific regions 180 that have been identified are then anonymized with replacement images 152, using a machine learning model 320, to obfuscate the identifying information to define a savable image 90 (step 608). The savable image 90 is then used for applying the replacement images 152 to the remaining images of the real-time video stream to define a savable image 90 in the form of a savable video (step 610). The savable video is then stored within the anonymized database 94 (step 612). The anonymized database 94 can then be categorized based upon training categories 210 (step 614). It is contemplated that a particular savable image 90 may be categorized within a number of separate training categories 210 within the anonymized database 94. By way of example, and not limitation, a particular video may include a number of different occurrences or scenarios that may be pertinent to different components or modules 212 of training and education. Accordingly, these videos may be stored or referenced within a number of training categories 210 within the anonymized database 94.

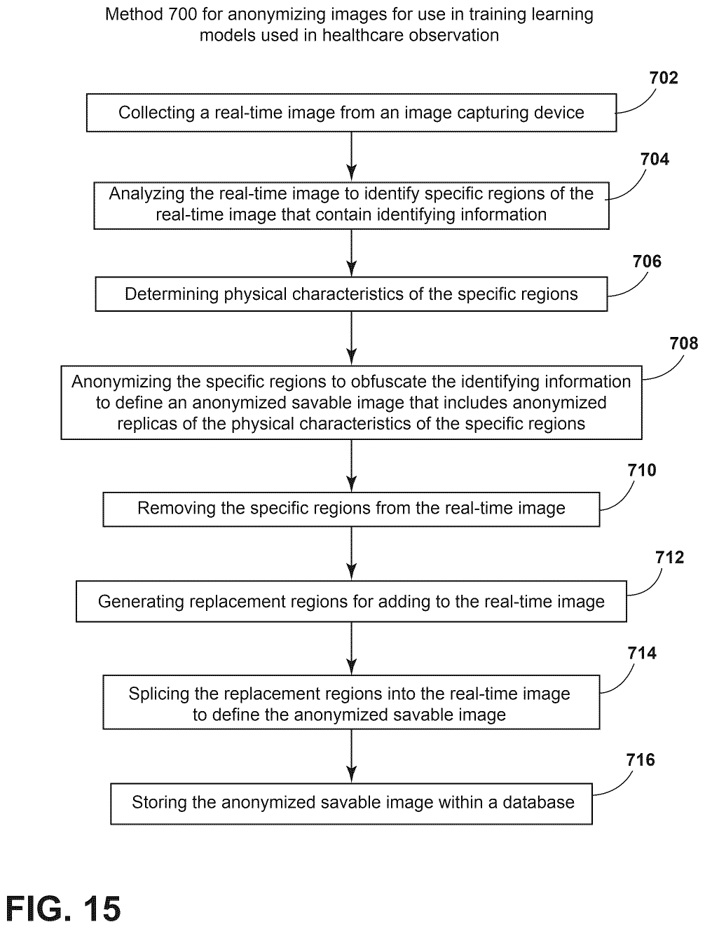

Referring now to FIGS. 1-9 and 15, having described various aspects of the device, a method 700 is disclosed for anonymizing images for use in training learning models 320 used in healthcare observation. According to the method 700, step 702 includes collecting a real-time image 26 from an image capturing device 18. The real-time image 26 is then analyzed to identify specific regions 180 of the real-time image 26 that contain identifying information (step 704). This identifying information could take the form of PHI 120 and/or PII 122, as well as information that can lead to the retrieval of PHI 120 and/or PII 122. Once the specific regions 180 are identified, the physical characteristics 350 of the specific regions 180 are determined (step 706). This determination of physical characteristics 350 can be accomplished through a machine learning model 320 that utilizes masks 250 or filters for segmenting the pixels 252 and other data of the real-time image 26, as described herein. According to the method 700, a step 708 includes anonymizing the specific regions 180 to obfuscate the identifying information to determine the anonymized savable image 90. This savable image 90, as described herein, includes anonymized replicas 286 of the physical characteristics 350 of the specific regions 180. As described herein, the physical characteristics 350 can include the silhouette 282 and the internal boundaries 284 of the specific regions 180. This anonymizing step can include a step 710 of removing the specific regions 180 from the real-time image 26. Once removed, replacement regions 280 are generated for adding to the real-time image 26 (step 712). The replacement regions 280 include the anonymized replicas 286 of the physical characteristics 350. These anonymized replicas 286 of the physical characteristics 350 can include a similar silhouette 288 and similar boundaries 290, as compared to the silhouette 282 and internal boundaries 284 of the specific regions 180 from the real-time image 26. The replacement regions 280 are then spliced into, or overwritten into, the specific regions 180 of the real-time image 26 to define the anonymized savable image 90 (step 714). The anonymized savable image 90 is then stored within the database 94 (step 716).

As described herein, the replacement regions 280 can be overwritten into the data of the real-time image 26 to render the specific regions 180, and the PHI 120 and PII 122 contained therein, irretrievable. It is also contemplated that the specific regions 180 can be redacted or deleted from the real-time image 26 before the replacement regions 280 are added back into the sections of data left by the removal of the specific regions 180. A combination of these approaches can also be utilized where certain information is overwritten and certain information is deleted and replaced.

According to the various aspects of the device, the silhouette 282 and the internal boundaries 284 correspond to the physical characteristics 350 of the specific regions 180. The similar silhouette 288 and the similar boundaries 290 defined by the anonymized replicas 286 of the physical characteristics 350 can be used to create the anonymized savable image 90 that can be stored within the database 94. Additionally, it is typical that the silhouette 282 and internal boundaries 284, as well as the similar silhouette 288 and the similar boundaries 290, correspond to portions of the human body 182. As described herein, the anonymized savable images 90 are compared against the subsequent video feed using a machine learning model 320. Within this machine learning model 320, the anonymized savable image 90 is used in relation to the subsequent video feed for detecting and identifying the presence of individuals within the subsequent video feed.

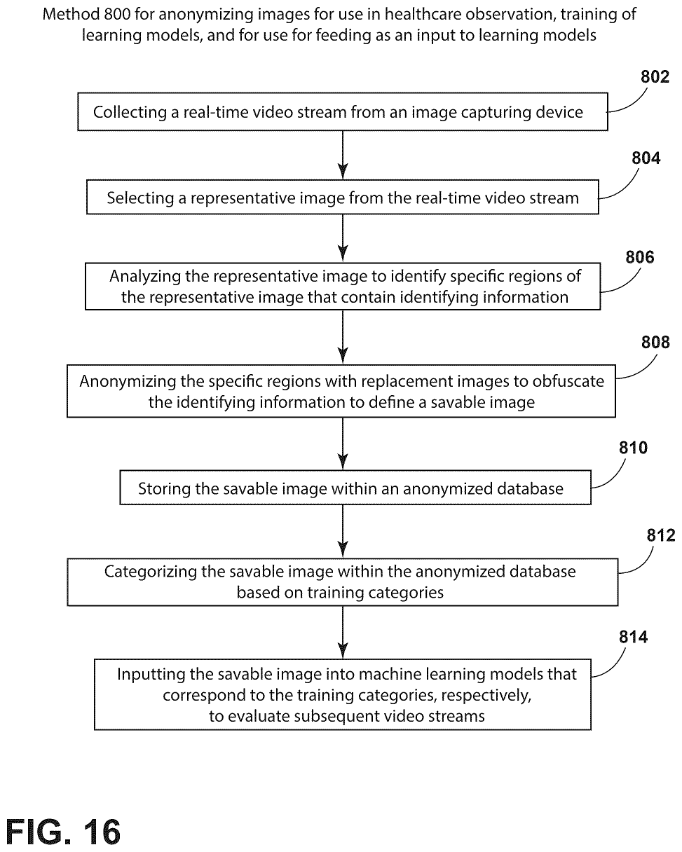

Referring now to FIGS. 1-9 and 16, having described various aspects of the device, a method 800 is disclosed for anonymizing images for use in healthcare observation, training of learning models 320, and for use in feeding as an input to machine learning models 320. According to the method 800, a step 802 includes collecting a real-time video stream from the image capturing device 18. A representative image 310 is then selected from the real-time video stream (step 804). As described herein, this representative image 310 can be a randomly selected or captured image, or can be an image discriminately selected from the real-time video stream as containing certain desirable indicators to include within the eventually derived savable image 90. The representative image 310 is then analyzed to identify specific regions 180 that contain identifying information (step 806). The specific regions 180 are then anonymized with replacement images 152 to obfuscate the identifying information to define the savable image 90 (step 808). The savable image 90 is then stored within an anonymized database 94 (step 810). The savable image 90 can be categorized within the anonymized database 94 based upon certain training categories 210 (step 812). These training categories 210 can include, but are not limited to, patient events, staff events, medical diagnoses, facility and staff operational efficiency, false alarms, combinations thereof, and other similar categories that can be used for evaluating the savable image 90, as described herein. The savable image 90 is then inputted into the machine learning model 320 that corresponds to the training category 210, respectively, to evaluate a subsequent video stream (step 814).

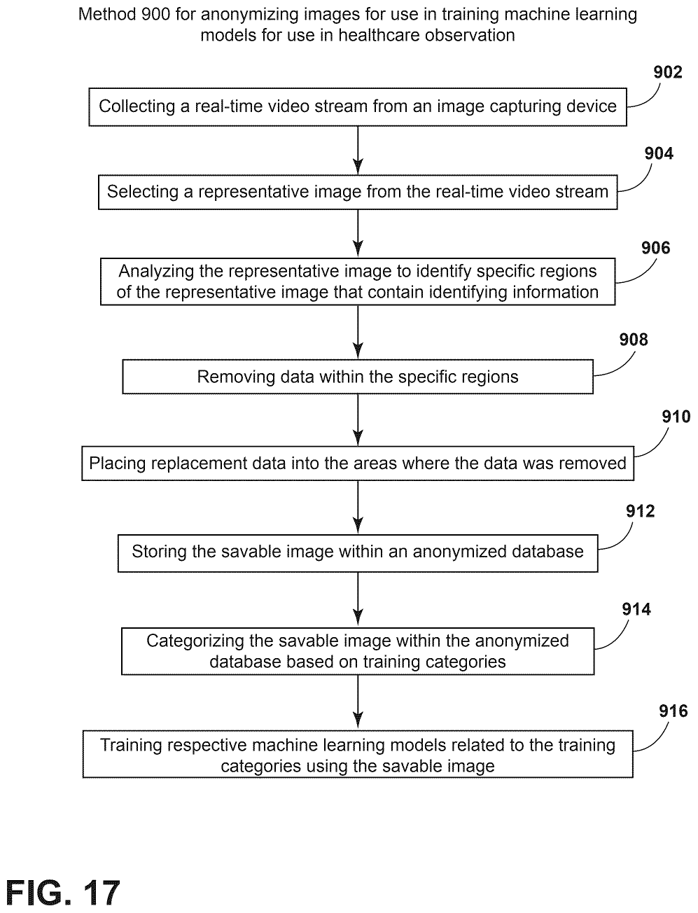

Referring now to FIGS. 1-9 and 17, having described various aspects of the device, a method 900 is disclosed for anonymizing images for use in training machine learning models 320 for use in healthcare observation. According to the method 900, a step 902 includes collecting a real-time video stream from an image capturing device 18. A representative image 310 is then selected from the real-time video stream (step 904). The representative image 310 is then analyzed to identify specific regions 180 of the representative image 310 that contain identifying information (step 906). The data within the specific regions 180 is then removed or overwritten (step 908). Replacement data is then placed into areas where the data was removed or is overwritten onto the data within specific regions 180, thereby obfuscating the identifying information to define the savable image 90 (step 910). The replacement data is at least partially derived from image data that is located within the representative image 310 and outside of the specific regions 180. As described herein, this use of image data from the representative image 310 is used to create the low contrast boundaries 340 or subtle contrast boundaries with respect to regions on the edge of the specific regions 180 of the savable image 90. The savable image 90 is then stored within the anonymized database 94 (step 912). The savable image 90 can be categorized within the anonymized database 94 based upon training categories 210 (step 914). As described herein, these training categories 210 can include patient safety, staff safety, medical diagnoses, facility and staff operational efficiency, false alarms, combination thereof, and other similar categories. A respective machine learning model 320 is then trained in a manner related to the training categories 210 using the savable image 90 (step 916).

As described herein, the savable image 90 is utilized by the machine learning model 320 for comparing and analyzing against individual frames of the real-time video stream. Commonalities between these two can provide data regarding the probability or probabilities of a particular event or set of events occurring in real-time or potentially occurring in the future. As the real-time video is analyzed further by the machine learning model 320, additional indicators can be gathered to provide for certain probabilities that certain events are, or are not, likely to occur. Over time, these probabilities can become more refined and reliable. Adding additional savable images 90 to the various training categories 210 of the anonymized database 94 provides for a more robust data set that can be compared against a real-time video stream for developing these probabilities of the various training categories 210 of events, as described herein.

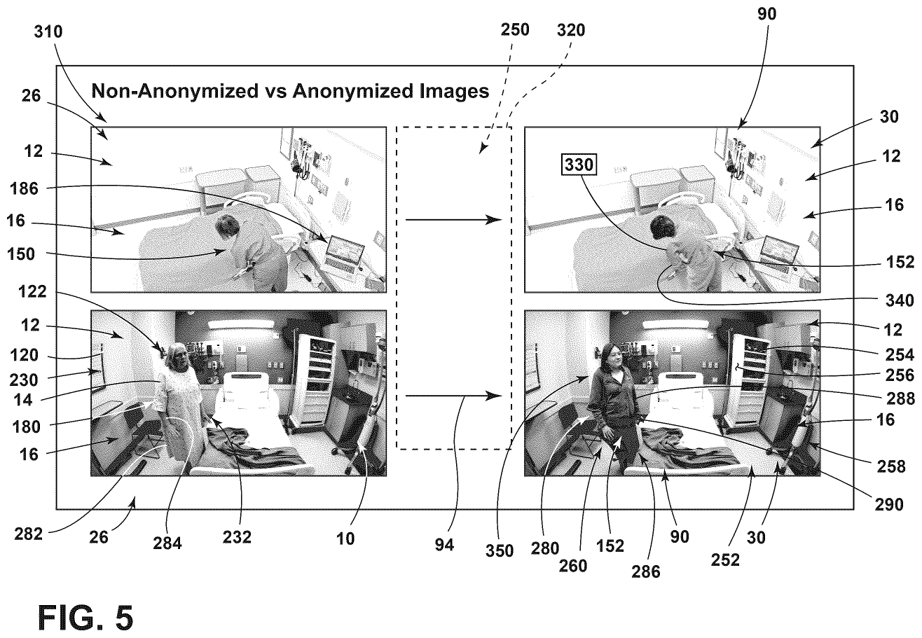

According to various aspects of the device, as exemplified in FIGS. 5-9, various training categories 210 can be included within the database 94. As exemplified in FIG. 5, the real-time image 26 appears on the left and the anonymized savable image 90 appears on the right. These images show that a particular healthcare setting 12 has been serviced by an orderly, or other member of the healthcare team, or that a healthcare professional has made the appropriate rounds or patient visits. Accordingly, the anonymization of the real-time images 26 can be saved into these savable images 90 for training the AI machine learning model 320 regarding events that may take place within the healthcare space 16. The savable images 90 can also be used for providing training with respect to monitoring healthcare staff and professionals and their movements within a particular healthcare space 16.

According to the various aspects of the device, the savable images 90 can also show movements of the patient 14 within the healthcare space 16, where these movements are authorized or unauthorized, such as in the case of a patient 14 designated as a fall risk. Also, within these representative images 310 from the real time image 26, various PII 122 and PHI 120 can be present, such as a whiteboard containing certain information, a laptop with a video screen facing the image capturing device 18, the facial features 184 and body 182 of the patient 14, the facial features 184 and body 182 of an occupant 150 or member of the healthcare team, and other similar PHI 120 and PII 122. Each of these portions of the image represents specific regions 180 that may contain PHI 120 or PII 122 that is to be obfuscated using the anonymizing system 92 by including blurring and replacement images 152 to obfuscate this information.

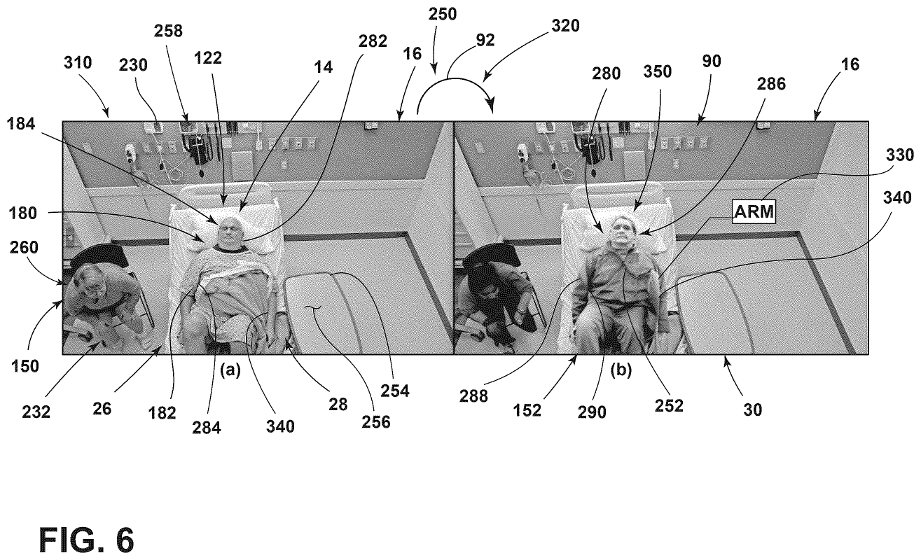

As exemplified in FIG. 6, the real-time image 26 illustrated in (a) shows the raw data from the image capturing device 18. The savable image illustrated in (b) shows the resulting savable image 90 after the anonymizing system 92 has performed the analysis and anonymization steps to produce the savable image 90. As exemplified in FIG. 6, the savable image 90 can be used with respect to training for identifying the patient 14 in a stable condition 28 within the bed.

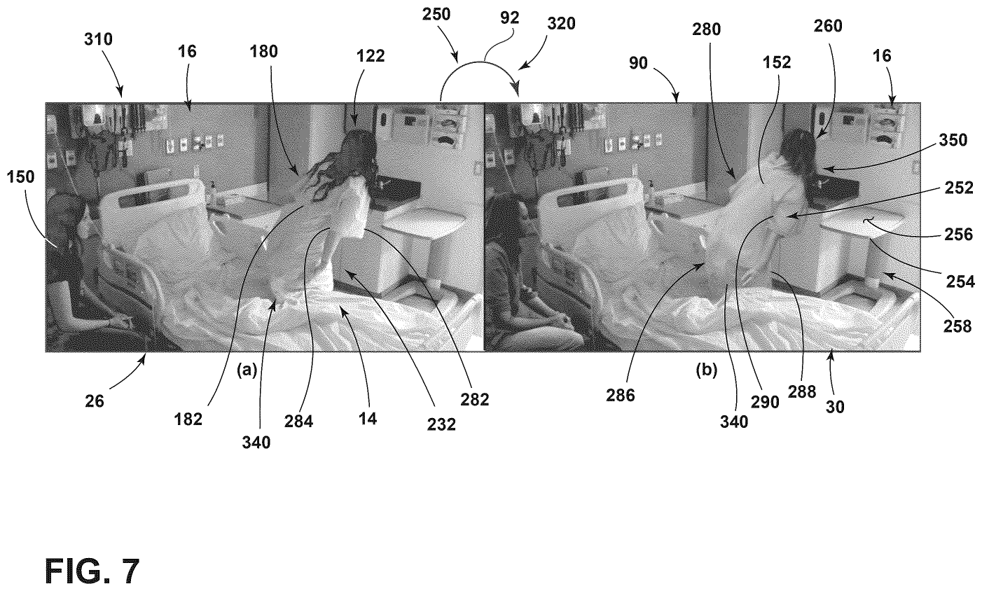

As exemplified in FIG. 7, the real-time image 26 illustrated in (a) is shown with the patient 14 getting up from the bed. The savable image 90 illustrated in (b) shows the same motions of the patient 14 with the PII 122 and PHI 120 anonymized and obfuscated so that the identity of the patient 14 cannot be ascertained. The image and likeness of the other occupant 150 of the room is also obfuscated. The image shown in FIG. 7 may be used for training a machine learning model with respect to an elopement situation or a patient 14, designated as being a fall risk, getting up from bed.

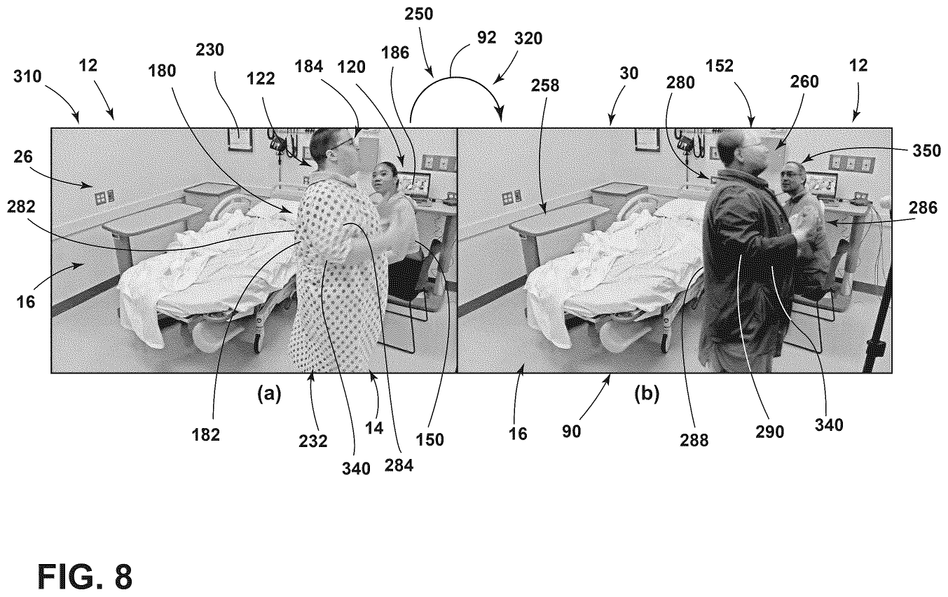

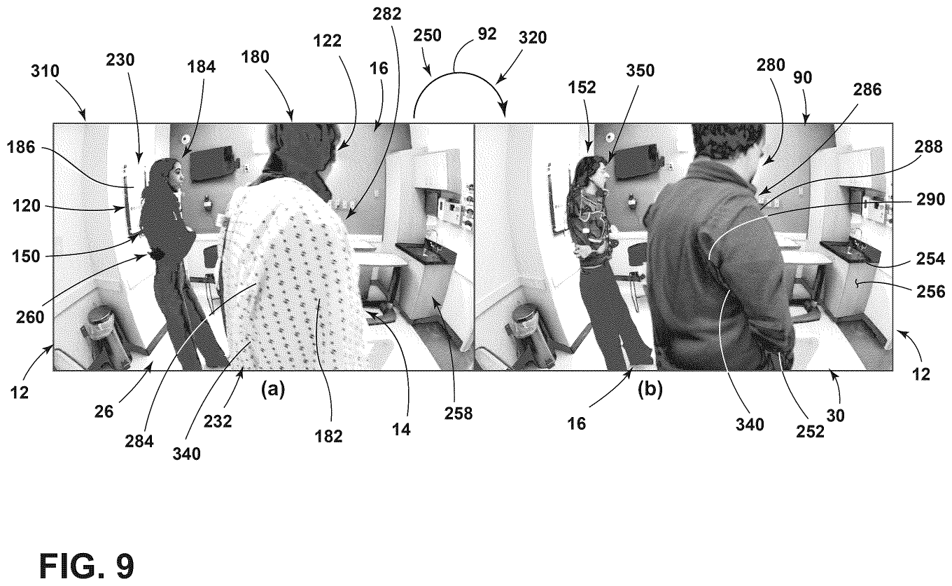

Referring now to FIGS. 8-9, FIG. 8(a) and FIG. 9(a) each show the raw real-time image 26 showing the patient 14 moving around the healthcare space 16 speaking with another occupant 150 of the healthcare space 16. The PHI 120 and PII 122 within this real-time image 26 is obfuscated to produce the savable image 90 shown in FIG. 8(b) and FIG. 9(b), respectively, with the image and likeness of the patient 14 and the occupant 150 obfuscated and other images showing PII 122 and PHI 120 also obfuscated to prevent the storage of sensitive information. Such images may be useful for training to recognize abusive speech or other adverse interaction between the patient 14 and the occupant 150.

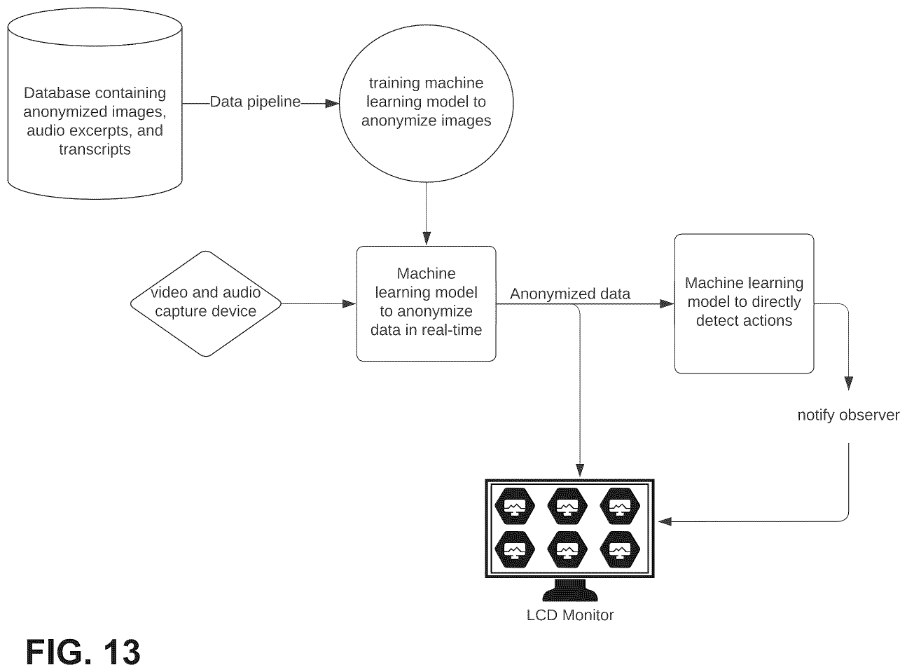

Referring now to FIG. 13, the database 94 includes anonymized savable images 90 and other anonymized audio-visual data that can be utilized by a machine learning model 320. This anonymized audio-visual data can include excerpts of information, transcripts of information and other forms of information. The machine learning model 320 is attached to the database 94 and draws from the database 94 to compare saved images with real-time images 26 that are captured by an image capturing device 18. The machine learning model 320 can utilize this database 94 for assisting an observer 24 in monitoring a patient 14. The machine learning model 320 can also analyze real-time images 26 to provide more robust anonymization of the specific regions 180 of the real-time images 26. Accordingly, the machine learning model 320 can be used to continuously gather additional anonymized data for adding to the database 94. As the database 94 builds, the anonymized data within the database 94 can be used to provide a more robust AI machine learning model 320 that can more efficiently monitor, analyze, and evaluate subsequent video feeds and other real-time images 26 for observation of a patient 14. Through this process, the machine learning model 320 can become more robust over time by adding additional anonymized data to the database 94. This continuously more robust database 94 also creates an ambient observation model that can passively monitor a patient 14 to assess and predict adverse events that may take place within a healthcare space 16. Accordingly, the machine learning model 320 can assist an observer 24 in monitoring a patient 14 and, in certain settings, can provide ambient monitoring of a patient 14 within a healthcare space 16.

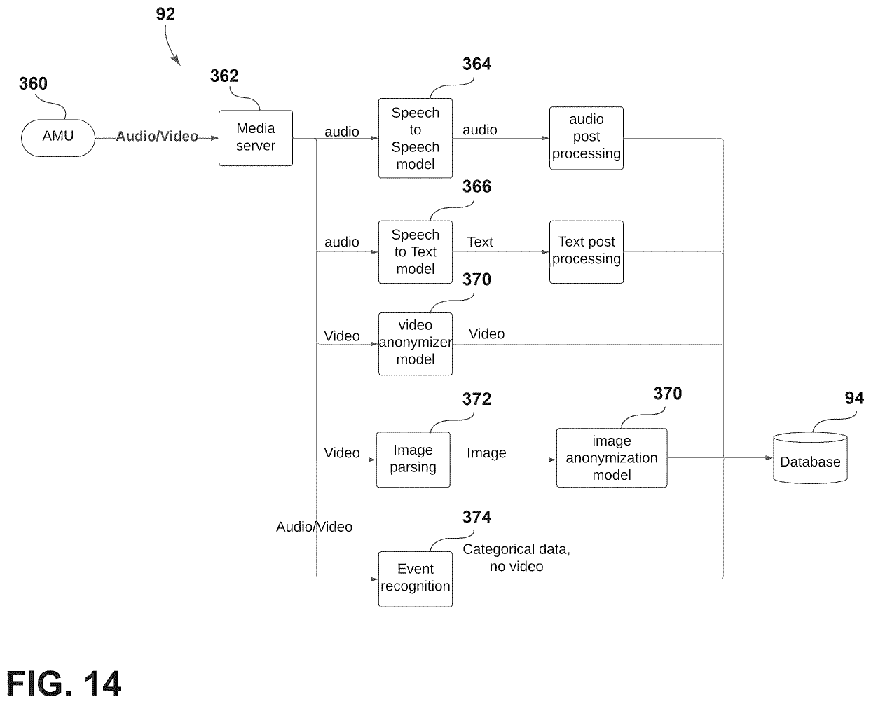

Referring now to FIG. 14, a schematic diagram is illustrated for describing an aspect of the anonymizing system 92 which can be used to anonymize various types of media including audio files, video files, and audio/video files. The AMU 360 (Active Monitoring Unit) directs media files to a media server 362. The media server 362 then delivers the corresponding media file to be processed and analyzed within one or more of various learning models that can correspond to media types delivered by the AMU 360. Where an audio file is delivered, the media server 362 delivers the audio file to a speech-to-speech learning model 364 for anonymization. This audio file is then delivered to a database 94 after post-processing is used for anonymizing the audio, as described herein. It is also contemplated that an audio file can be delivered to a speech-to-text learning model 366 for anonymization. After processing, a text file is then delivered to a database for use by a machine learning model 320.

Referring again to FIG. 14, where a video file is delivered by the media server 362, the video file can be delivered to an anonymizing learning model 370 for performing the obfuscation operation on the video file. Alternatively, the video file can be delivered to an image parsing learning model 372. The image parsing learning model 372 can be used to perform the process of segmentation, as described herein. Once the process of segmentation is complete, the now segmented and parsed video file can be delivered to the anonymizing learning model 370 to be obfuscated with respect to representative images of the video file or sections of the video file. In certain aspects of the device, the audio/video file can be delivered to an event recognition learning model 374 where the learning model 320 analyzes the audio/video file to determine an event that has occurred within the file. In this aspect of the device, categorical data can be produced for delivering to the database 94. This categorical data can then be stored within the database 94 for use by the machine learning model 320 with respect to subsequent video feeds.

As exemplified in FIG. 14, a range of learning models 320 can be utilized for processing and analyzing a range of media types and media files. Additionally, the range of learning models 320 can be used in combination to process, analyze, and obfuscate different portions (audio, video, text, and the like) of a single media file. Using these various learning models 320, the representative images 310 can be reviewed by a number of dedicated learning models 320 for removing the PHI 120 and the PII 122 before the image is stored within the database 94. Additionally, using the range of learning models 320, various information can be annotated using the tags 330 for developing a usable savable image 90 that does not include PHI 120 or PII 122, but is usable by the learning model 320 in reviewing subsequent video feeds.

According to the various aspects of the device, the anonymizing system 92 described herein is utilized to create a database 94 of anonymized savable images 90 that can be used for training and education purposes. In particular, the anonymizing system 92 uses one or more machine learning models 320 to identify those specific regions 180 of the real-time image 26 that contain PHI 120 and PII 122. The one or more machine learning models 320 are configured to distinguish certain portions of the real-time image 26 that contain this sensitive information from those portions of the real-time image 26 that contain innocuous information. These innocuous portions of the real-time image 26 do not contain sensitive information, but may be useful in training an observer 24 or educating an observer 24. Additionally, the one or more machine learning models 320 use the replacement images 152 to retain the movements of the patient 14 and the occupants 150, without retaining any sensitive information that may contain, or lead to, PHI 120 or PII 122. The one or more machine learning models 320 accomplish this analysis of the real-time image 26 and the anonymization of the specific regions 180 of the real-time image 26 that contain PHI 120 or PII 122 without the aid of an individual. As discussed herein, it may be desired to have a confirmation step conducted by the machine learning model 320, or by a separate program, or an individual to confirm that the PHI 120 and the PII 122 has been properly anonymized before saving the savable image 90 to the database 94.

The training and education of observers 24 is desired so that the observer 24 can be equipped to address a wide range of situations within a particular healthcare space 16 that is being observed. Use of the anonymizing system 92 allows for the creation of this database 94 to include real-world representations of events that have actually occurred, without storing or disclosing PHI 120 or PII 122. Accordingly, the anonymized database 94 of categorized savable images 90 provides a robust set of data that can be used by new observers 24 as well as experienced observers 24 to gain training and continuing education with respect to their patient observation duties.

According to one aspect of the present disclosure, a method for anonymizing images for use in training learning models used in healthcare observation, wherein the images can be used as input data for the learning models, includes steps of collecting a real-time image from an image capturing device, analyzing the real-time image to identify specific regions of the real-time image that contain identifying information, determining physical characteristics of the specific regions, anonymizing the specific regions to obfuscate the identifying information to define an anonymized savable image that includes anonymized replicas of the physical characteristics of the specific regions, and storing the anonymized savable image within a database.

According to another aspect, the step of anonymizing the specific regions includes removing the specific regions from the real-time image, generating replacement regions for adding to the real-time image, the replacement regions including the anonymized replicas of the physical characteristics, and splicing the replacement regions into the real-time image to define the anonymized savable image.

According to another aspect, the real-time image is a randomly captured image that includes at least a patient, and the specific regions include portions of the patient.

According to another aspect, the real-time image is selected from a video feed that includes an identified event.

According to another aspect, the replacement regions correspond to sections of the specific regions having a silhouette and internal boundaries. The replacement regions include a similar silhouette and similar boundaries as compared to the silhouette and internal boundaries of the specific regions.

According to another aspect, the silhouette and the internal boundaries correspond to the physical characteristics of the specific regions, and the similar silhouette and the similar boundaries define the anonymized replicas of the physical characteristics.

According to another aspect, the silhouette and the internal boundaries correspond to portions of a human body.

According to another aspect, the anonymized savable image is compared to subsequent real-time images at least for detecting and identifying a presence of individuals within the subsequent real-time image.

According to another aspect, the step of anonymizing the specific regions of the real-time image includes applying tags to the real-time image. The tags are applied at least to the replacement regions to define the anonymized savable image.

According to another aspect, the anonymized savable image is stored within the database according to training model categories of potential events.

According to another aspect, the training model categories include patient events, staff events, medical diagnosis, facility and staff operational efficiency, and false alarms.

According to another aspect of the disclosure, a method for anonymizing images for use in healthcare observation, training of learning models, and for use for feeding as an input to learning models includes steps of collecting a real-time video stream from an image capturing device, selecting a representative image from the real-time video stream, analyzing the representative image to identify specific regions of the representative image that contain identifying information, anonymizing the specific regions with replacement images to obfuscate the identifying information to define a savable image, storing the savable image within an anonymized database, categorizing the savable image within the anonymized database based on training categories, and inputting the savable image into machine learning models that correspond to the training categories, respectively, to evaluate subsequent video streams.

According to another aspect, the training categories include conducting observation related to patient safety, staff safety, medical diagnosis, facility and staff operational efficiency, and false alarms.

According to another aspect, the step of anonymizing the specific regions includes removing the specific regions from the representative image, generating replacement regions for adding to the representative image, the replacement regions including anonymized replicas of physical characteristics, and splicing the replacement regions into the representative image to define the savable image.

According to another aspect, the replacement regions are at least partially generated using data from regions of the representative image outside of the specific regions.

According to another aspect, the replacement regions are at least partially generated using data from sections of the representative image outside of the specific regions and along edges of the specific regions. The data is used to generate the replacement regions having subtle contrast boundaries with respect to the sections outside the specific regions.

According to another aspect, the replacement regions correspond to sections of the specific regions having a silhouette and internal boundaries. The replacement regions include a similar silhouette and similar boundaries as compared to the silhouette and internal boundaries of the specific regions.

According to another aspect, the silhouette and the internal boundaries correspond to the physical characteristics of the specific regions, and the similar silhouette and the similar boundaries define the anonymized replicas of the physical characteristics.

According to another aspect, the silhouette and the internal boundaries correspond to portions of a human body.

According to yet another aspect of the disclosure, a method for anonymizing images for use in training machine learning models for use in healthcare observation includes steps of collecting a real-time video stream from an image capturing device, selecting a representative image from the real-time video stream, analyzing the representative image to identify specific regions of the representative image that contain identifying information, removing data within the specific regions, placing replacement data into areas where the data was removed, thereby obfuscating the identifying information to define a savable image, wherein the replacement data is at least partially derived from image data that is located within the representative image and outside of the specific regions, storing the savable image within an anonymized database, categorizing the savable image within the anonymized database based on training categories, wherein the training categories include patient safety, staff safety, medical diagnosis, facility and staff operational efficiency, and false alarms, and training respective machine learning models related to the training categories using the savable image.

It will be understood by one having ordinary skill in the art that construction of the described disclosure and other components is not limited to any specific material. Other exemplary embodiments of the disclosure disclosed herein may be formed from a wide variety of materials, unless described otherwise herein.

What is claimed is:

1. A method for anonymizing images for use in training learning models used in healthcare observation, wherein the images can be used as input data for the learning models, the method comprising steps of:
collecting a real-time image from an image capturing device;
analyzing the real-time image to identify specific regions of the real-time image that contain identifying information;
determining physical characteristics of the specific regions;
anonymizing the specific regions to obfuscate the identifying information to define an anonymized savable image that includes anonymized replicas of the physical characteristics of the specific regions; and
storing the anonymized savable image within a database.

2. The method of claim 1, wherein the step of anonymizing the specific regions comprises:
removing the specific regions from the real-time image;
generating replacement regions for adding to the real-time image, the replacement regions including the anonymized replicas of the physical characteristics; and
splicing the replacement regions into the real-time image to define the anonymized savable image.

3. The method of claim 1, wherein the real-time image is a randomly captured image that includes at least a patient, and wherein the specific regions include portions of the patient.

4. The method of claim 1, wherein the real-time image is selected from a video feed that includes an identified event.

5. The method of claim 2, wherein the replacement regions correspond to sections of the specific regions having a silhouette and internal boundaries, wherein the replacement regions include a similar silhouette and similar boundaries as compared to the silhouette and internal boundaries of the specific regions.

6. The method of claim 5, wherein the silhouette and the internal boundaries correspond to the physical characteristics of the specific regions, and wherein the similar silhouette and the similar boundaries define the anonymized replicas of the physical characteristics.

7. The method of claim 6, wherein the silhouette and the internal boundaries correspond to portions of a human body.

8. The method of claim 1, wherein the anonymized savable image is compared to subsequent real-time images at least for detecting and identifying a presence of individuals within the subsequent real-time image.

9. The method of claim 2, wherein the step of anonymizing the specific regions of the real-time image includes applying tags to the real-time image, wherein the tags are applied at least to the replacement regions to define the anonymized savable image.

10. The method of claim 1, wherein the anonymized savable image is stored within the database according to training model categories of potential events.

11. The method of claim 10, wherein the training model categories include patient events, staff events, medical diagnosis, facility and staff operational efficiency, and false alarms.

12. A method for anonymizing images for use in healthcare observation, training of learning models, and for use for feeding as an input to learning models, the method comprising steps of:
collecting a real-time video stream from an image capturing device;
selecting a representative image from the real-time video stream;
analyzing the representative image to identify specific regions of the representative image that contain identifying information;
anonymizing the specific regions with replacement images to obfuscate the identifying information to define a savable image;
storing the savable image within an anonymized database;
categorizing the savable image within the anonymized database based on training categories; and
inputting the savable image into machine learning models that correspond to the training categories, respectively, to evaluate subsequent video streams.

13. The method of claim 12, wherein the training categories include conducting observation related to patient safety, staff safety, medical diagnosis, facility and staff operational efficiency, and false alarms.

14. The method of claim 12, wherein the step of anonymizing the specific regions comprises:
removing the specific regions from the representative image;
generating replacement regions for adding to the representative image, the replacement regions including anonymized replicas of physical characteristics; and
splicing the replacement regions into the representative image to define the savable image.

15. The method of claim 14, wherein the replacement regions are at least partially generated using data from regions of the representative image outside of the specific regions.

16. The method of claim 14, wherein the replacement regions are at least partially generated using data from sections of the representative image outside of the specific regions and along edges of the specific regions, wherein the data is used to generate the replacement regions having subtle contrast boundaries with respect to the sections outside the specific regions.

17. The method of claim 14, wherein the replacement regions correspond to sections of the specific regions having a silhouette and internal boundaries, wherein the replacement regions include a similar silhouette and similar boundaries as compared to the silhouette and internal boundaries of the specific regions.

18. The method of claim 17, wherein the silhouette and the internal boundaries correspond to the physical characteristics of the specific regions, and wherein the similar silhouette and the similar boundaries define the anonymized replicas of the physical characteristics.

19. The method of claim 18, wherein the silhouette and the internal boundaries correspond to portions of a human body.

20. A method for anonymizing images for use in training machine learning models for use in healthcare observation, the method comprising steps of:
collecting a real-time video stream from an image capturing device;
selecting a representative image from the real-time video stream;
analyzing the representative image to identify specific regions of the representative image that contain identifying information;
removing data within the specific regions;
placing replacement data into areas where the data was removed, thereby obfuscating the identifying information to define a savable image, wherein the replacement data is at least partially derived from image data that is located within the representative image and outside of the specific regions;
storing the savable image within an anonymized database;
categorizing the savable image within the anonymized database based on training categories, wherein the training categories include patient safety, staff safety, medical diagnosis, facility and staff operational efficiency, and false alarms; and
training respective machine learning models related to the training categories using the savable image.
